(19) United States
(12) Patent Application Publication
(10) Pub. No.: US 2025/0276022 A1
(43) Pub. Date: Sep. 4, 2025

(54) DENTIN MATRIX PROTEIN MATERIALS AND ASSOCIATED METHODS

(71) Applicants: Oregon Health & Science University, Portland, OR (US); Trustees of Tufts College, Medford, MA (US)

(72) Inventors: Luiz E. BERTASSONI, Portland, OR (US); Cristiane MIRANDA FRANÇA, Portland, OR (US); Jack L. FERRACANE, Beaverton, OR (US); Ashley SERCIA, Portland, OR (US); Anthony TAHAYERI, Sherwood, OR (US); Pamela C. YELICK, Boston, MA (US)

(21) Appl. No.: 18/857,576

(22) PCT Filed: Apr. 21, 2023
(86) PCT No.: PCT/US2023/066091

§ 371 Date: Oct. 17, 2024

Related U.S. Application Data
(60) Provisional application No. 63/334,052, filed on Apr. 22, 2022.
(60) Provisional application No. 63/363,452, filed on Apr. 22, 2022.

(51) Int. Cl. A61K 35/32; A61K 9/06; A61K 38/39; A61L 27/36; A61L 27/52

(52) U.S. Cl. CPC A61K 35/32; A61K 9/06; A61K 38/39; A61L 27/3608; A61L 27/52

(57) ABSTRACT

The present disclosure describes compositions, kits and methods for promoting regeneration of dental tissue, particular dental pulp tissue. In some embodiments, a composition for promoting regeneration of dental tissue includes a dentin extracellular matrix material (dECM). In some embodiments, the dECM comprises dentin matrix proteins having an origin selected from human, ovine, porcine, and bovine.

### FEDERALLY SPONSORED RESEARCH OR DEVELOPMENT

This invention was made with government support under Grant Nos. R01DE026170-01 and 5U24DE026915-02 awarded by the National Institutes of Health (NIH); and NIH/National Institute of Dental and Craniofacial Research (NIDCR) Grant No. 5U24DE029462-03, SUBK00016982. The government has certain rights in the invention.

### TECHNICAL FIELD

The present disclosure relates generally to regenerative dentistry. More specifically, the present disclosure relates to materials and methods for promoting the regeneration of dental tissue.

### BACKGROUND INFORMATION

Dental pulp is the innervated, unmineralized connective tissue that occupies a chamber in the center of the tooth that is surrounded by mineralized tissue, spanning from the root apex through the crown. The formation of dentin, the tissue surrounding the pulp, is achieved by odontoblasts, which are specialized cells that are located in a pseudo-stratified layer at the periphery of the pulp chamber. Among other tissue components, such as fibroblasts, neurons, and resident stem cells, the pulpal tissue comprises a network of blood capillaries that traverse centrally through the pulp extending towards the tooth crown. Microcapillaries branching outwards from the core vessel form a capillary-rich plexus a few micrometers away from the odontoblast layer near the dentin.

Root canal treatment is necessary in the event of deep caries or trauma when the homeostasis of the pulp tissue is lost. Current root canal treatment methods typically involve removal of infected or necrotic tissue and replacement with inert synthetic biomaterials, thus sacrificing the vitality of the tooth so as to render it brittle and more susceptible to fracture. Regeneration of the pulp tissue to restore tooth function, a strategy that has been named regenerative endodontics, has been proposed as an alternative to conventional root canal therapy. Regenerative endodontic approaches have focused on the transplantation of cells such as stem cells into the tooth to initiate the formation of pulp-like tissues and revascularization. However, successfully isolating, manipulating, and maintaining stem cells for transplantation in a clinical setting is challenging, and the seeded cells and growth factors may not exhibit the level of tissue specificity needed for regenerating dental pulp. This presents a significant hurdle to implementation of regenerative therapy in clinical practice. Thus, there remains a need for effective strategies that allow for controlled regeneration of vascularized pulp and can be readily implemented by clinicians.

### BRIEF DESCRIPTION OF THE DRAWINGS

FIG. 1 depicts tooth procurement and processing steps. Panel A is an image of teeth that were extracted and preserved in chloramine T for 3 hours until processing. Panel B is an image of teeth that were cleaned with a scalpel blade to remove soft tissue and sectioned with an automated saw to create slices. Panel C is an image of teeth which were ground to a powder in a freezer mill. Panel D is a graph showing weights of human and bovine teeth. Bovine teeth yielded 66% more powder than human teeth.

FIG. 2 depicts extraction of dentin matrix protein (DMP). Human dentin powder precipitates (panel A) and bovine dentin powder precipitates (panel B) are shown. Human dentin powder precipitates are smaller than bovine pellets. Panel C is a graph of proteins per tooth. Bovine dentin yielded more proteins per tooth than human dentin. Panels D and E are graphs showing daily protein extraction by solution A (panel D) and solution B (panel E). There was no difference in the daily protein extraction between Solution A and B.

FIG. 3A shows graphs depicting proteomic characterization of human and bovine dentin extracellular matrix material. Relative amounts of the 30 most abundant human dentin proteins and their respective orthologs in bovine dentin matrix extracts are shown. The top 30 proteins detected in human extracts have good orthologs with bovine extracts, but the relative proportion between species is different.

FIG. 3B shows graphs of relative amount of protein groups according to the biological family. Phosphorylated SIBLING proteins (left), structural proteins and proteoglycans (center), and growth factors (right) are shown. Bovine dentin matrix contains more osteocalcin, biglycan and insulin like growth factor 5 (IGF-5), whereas human dentin matrix presents higher amounts of osteopontin, osteomodulin, decorin, vitronectin and transforming growth factor beta 1 (TGFb-1).

FIG. 4 shows graphs of the number of cells in migration assays using transwell chambers. Results from methacrylated gelatin supplemented with either human (panel A) or bovine (panel B) proteins are shown.

FIG. 5 shows fluorescent images of cells seeded on bulk gels (panels A, B, C) or on microgels (panels D, E, F) stained for actin red. Top (panel A), middle (panel B), and bottom (panel C) of a bulk gel and top (panel D), middle (panel E), bottom (panel F) of a microgel are shown.

FIG. 6 shows fluorescent images of different compositions of methacrylated gelatin seeded with stem cells from apical papilla (SCAPs). Methacrylated gelatin supplemented with either 250 μg/ml dECM (panels A and E) or 500 μg/ml (panels B and F), microgels supplemented with 500 μg/ml dECM (panels C and G), and methacrylated collagen supplemented with 500 μg/ml dECM (panels D and H) are shown. (Scale bar=100 microns.)

FIG. 7 is a graph of fraction of cells over invasion distance in methacrylated gelatin, methacrylated collagen, and microgel.

FIG. 8 shows graphical representations and images of microgels. Graphical representations and images of microgel shape XY designed in Fusion 360 (Autodesk) (panel A), determination of hydrogel thickness by glass template thickness (panel B), individual microgel (panel C), placement of microgel designs in array form to pre-portioned application doses (panel D), removal of excess hydrogel from printed array and collection for lyophilization (panels E and F) are shown.

FIG. 9 shows percentages of cells on top of membrane surface and bottom side of membrane in methacrylated gelatin, DMP-supplemented methacrylated gelatin, platelet-derived growth factor (PDGF), and MTA-treated groups.

FIG. 10 is a graph showing MTT absorbance results in a metabolic activity assay. Methacrylated gelatin supplemented with bovine DMP did not show a significant loss of metabolic activity compared to unsupplemented methacrylated gelatin or platelet-derived growth factor (PDGF; positive control).

FIG. 11 shows representative images of a pulp capping procedure. From left to right: representative image of dentin matrix protein-supplemented gelatin methacryloyl (also referred to herein as RegendoGEL™), placement of RegendoGEL into a cavity, hydration of RegendoGEL, placement of thin layer of MTA, and sealing and restoration of cavity are shown.

FIG. 12 shows representative histology images showing the effect of different pulp capping materials on the morphology and organization of dental pulp in a pulpotomy rat model. Each pair of micrographs exhibits a cross-sectional view of a representative sample of each group in low and higher magnifications. The red arrowhead depicts the location of the defect in each tooth. Left side panels A, B: negative control; panels C, D: MTA; panels E, F: microgel+PDGF 10 ng/ml; panels G, H: Microgel; panels I, J: microgel+DMP 500 μg/ml; panels K, L: microgel+DMP 1000 μg/ml. The presence of newly formed dentin tertiary dentin is visible in all microgel containing groups (panels G-L).

FIG. 13 shows graphs of percentage of necrosis and tertiary dentin in the pulp chamber of the pulpotomy rat model. Panel A: Quantification of necrosis in the dental pulp tissue under the pulp capping treatment. Data is shown as percent area of the pulp chamber±S.D. Panel B: Quantification of tertiary dentin in the dental pulp tissue under the pulp capping treatment. Data is shown as percentual area of the pulp chamber±S.D.

FIG. 14 is a graph showing inflammation scores of dental pulp tissue 5 days post-placement.

FIG. 15 shows representative histology images (panels A, C, E) and microCT images (panels B, D, F) and graph (panel G) of mineral deposit thickness of dental pulps treated with RegendoGEL (panels A and B), GelMA (panels E and F) or MTA (panels C and D) within 5 days.

FIG. 16 shows representative histology images (panels A, C, E) and

microCT images (panels B, D, F) and graph (panel G) of mineral deposit thickness of dental pulps treated with RegendoGEL (panels A and B), GelMA (panels E and F) or MTA (panels C and D) within 70 days.

FIG. 17 shows representative images of in vivo experiments. Rehydrated lyophilized microgels unsupplemented or supplemented with DMP or PDGF in tubes are shown in panel A. Microgels placed into 1-mm thick dentin slices (panel B) and insertion into the subcutaneous pockets of immunocompromised rats (panel C) are also shown. Panels D and E show representative images of inserted microgels in rats at 5 days post implantation and one month post implantation, respectively.

FIG. 18 shows images of H&E stained dentin slices at 5 days after implantation into the subcutaneous pocket of immunocompromised rats. Methacrylated gelatin (upper left), methacrylated gelatin+PDGF (upper right), MTA (lower left) and unsupplemented gelatin (lower right) are shown.

FIG. 19 shows images of H&E stained dentin slices from root canal model treated with methacrylated gelatin at thirty days. Formation of odontoblast-like layer near the dentin, highly cellularized pulp-like tissue with extensive blood vessels (magnified region indicated by inset box) can be observed. Magnified image is also shown (right).

FIG. 20 shows images of RegendoGEL microparticles stored in Eppendorf tube (top), picked up with tweezers after adding one drop of saline solution (middle), and a human molar drilled to gain access to pulp chamber, cleaned, and filled with RegendoGEL microparticles. This process can also be applied to dog teeth.

FIG. 21 shows images of a RegendoGEL membrane pre- and post-lyophilization, including a 450 μm thick RegendoGEL membrane after light cure (top) and 450 μm thick RegendoGEL after 24-hr freeze dry and 24-hr lyophilization (bottom).

FIG. 22 shows images a thinner RegendoGEL membrane post-lyophilization, including a 350 μm thick RegendoGEL membrane after light cure, 24-hr freeze dry (top) and 24-hr lyophilization (bottom).

FIG. 23 shows images of a human molar access for membrane implantation, in which a human molar was drilled to gain access to pulp chamber, cleaned, and treated with RegendoGEL membrane.

FIG. 24 shows images of dental instruments used for RegnedoGEL membrane implantation (top), a RegendoGEL membrane picked up with tweezers for implantation of material (middle), and a dog molar drilled to gain access to pulp chamber, cleaned, and prepared for RegendoGEL membrane implantation (bottom).

### DETAILED DESCRIPTION OF EMBODIMENTS

In some embodiments, RegendoGEL is a stable, freeze-dried hydrogel microparticulate material that can be placed directly onto the dental pulp. It is composed of DMPs or dentin matrix molecules (DMMs) and a hydrogel carrier. DMPs are harvested from extracted bovine teeth, which are shown to yield more proteins per tooth than from human teeth, with comparable composition and efficacy. The inclusion of DMPs stimulates dental pulp cellular invasion and adhesion, promoting tissue regeneration in the dental pulp. DMPs also stimulate reparative dentin bridge formation to protect the dental pulp.

In accordance with an embodiment, a composition for promoting regeneration of dental tissue comprises a dentin extracellular matrix material (dECM) dispersed within a hydrogel carrier.

The dECM can be obtained from harvested dentin and may include one or more compounds selected to stimulate cellular invasion promoting tissue regeneration in the pulp of a tooth. In some embodiments, the dECM comprises one or more dentin matrix proteins (DMPs). The particular compound(s) provided by the dECM may depend upon the nature of the dentin from which the material is sourced. In some embodiments, the dECM comprises one or more human DMPs. In some embodiments, the dECM comprises one or more bovine DMPs.

In some embodiments, the dECM is present in the composition in a concentration of from about 100 μg/ml to about 1,200 μg/ml. In a more specific embodiment, the concentration is from about 250 μg/ml to about 600 μg/ml.

The composition can comprise a crosslinkable polymer material to serve as a carrier for the dECM. The polymer material may be selected to provide clinically useful properties, e.g., biocompatibility, biodegradability and photocurability. One example of this type of material include gelatins in which denatured collagen is functionalized to form photocrosslinkable precursors. In some cases, the physical properties of such materials upon curing, e.g., stiffness and porosity, may be tuned by manipulating the degree of functionalization. In some embodiments, the polymer material comprises a methacrylated gelatin such as gelatin methacryloyl (GelMA).

The polymer material may be self-curing and/or may be curable by other means, such as by light, temperature, chemical agents, or mechanical means. In some embodiments, the composition further comprises a crosslinking agent selected to facilitate crosslinking of the polymer material. In a more specific embodiment, the crosslinking agent is a photoinitiator. Suitable photoinitiators include, but are not limited to, lithium phenyl-2,4,6-trimethylbenzoyl phosphinate, lithium acylphosphinate, 2-Hydroxy-4′-(2-hydroxyethoxy)-2-methylpropiophenone and 2-hydroxy-1-(4-(hydroxyethoxy)phenyl)-2-methyl-1-propanone. The photoinitiator may be present in the composition at a concentration from about 0.05 wt % to about 2 wt %. In an embodiment, the composition comprises lithium acylphosphinate at a concentration from about 0.05 wt % to about 0.1 wt %. In some embodiments, the photoinitiator may be selected based upon the wavelengths of light by which it is activated. For example, in some embodiments, the photoinitiator can have an activation wavelength of from about 200 nm to about 500 nm. In some embodiments, the photoinitiator has an activation wavelength of from about 400 nm to about 700 nm.

In some embodiments, the composition comprises a polymer material that is at least partially crosslinked to provide a hydrogel material containing dECM. In some embodiments, the hydrogel material may be disaggregated into discrete microparticles or pieces. In particular embodiments, the hydrogel material may be lyophilized or freeze-dried to provide a microparticulate material that can be rehydrated to enable release of dECM from the material. In some embodiments, the composition comprises a polymer material, dECM (such as DMPs), and a cross-linking agent (optionally, a photoinitiator), and one or more components of the composition are crosslinked and/or the composition is “cured” prior to treating a subject (e.g., prior to applying the composition to a subject's tooth). In some embodiments, the crosslinked (“cured”) composition is disaggregated, cut, and/or shaped, prior to treatment of a subject. In some embodiments, the crosslinked (“cured”) composition is applied to a subject's tooth in the form of a sheet or droplets. In some embodiments, the composition is cured in a mold and is thereby shaped.

A method for making a composition as described herein can comprise providing dECM and combining the dECM with a polymer material that is crosslinkable, biocompatible and biodegradable. These can be further combined with a crosslinking agent such as a photoinitiator. The method can further comprise at least partially crosslinking the polymer material to produce a hydrogel. In some embodiments, the method comprises crosslinking the polymer material prior to treating a subject, (e.g., prior to applying the composition to the subject's tooth).

In some embodiments, providing dECM can comprise obtaining dentin, for example from harvested teeth. In particular embodiments, the dentin may be obtained from teeth of one or more origins including but not limited to, human, bovine, porcine and ovine. The dentin may then be processed to extract and isolate dECM including DMPs from the dentin. In some embodiments, the dECM may be stored for a time before use. This can comprise lyophilizing the dECM to produce a powder that may be stably stored at low temperature.

The dECM may be combined with a crosslinkable polymer material, such as a functionalized gelatin, so that the dECM is dispersed in the polymer material. In some embodiments, the polymer material comprises a methacrylated gelatin, for example, GelMA. In an embodiment, the amount of dECM is selected to provide a concentration of dECM in the composition from about 100 μg/ml to about 1,200 μg/ml. In a more specific embodiment, the concentration is from about 250 μg/ml to about 600 μg/ml.

As noted above, the dECM and the polymer material may additionally be combined with a crosslinking agent. In some embodiments, the polymer material comprises the crosslinking agent. In particular embodiments, the crosslinking agent is a photoinitiator. Suitable photoinitiators include, but are not limited to, lithium phenyl-2,4,6-trimethylbenzoyl phosphinate, lithium acylphosphinate, 2-Hydroxy-4′-(2-hydroxyethoxy)-2-methylpropiophenone and 2-hydroxy-1-(4-(hydroxyethoxy)phenyl)-2-methyl-1-propanone. The photoinitiator may be present in the composition at a concentration from about 0.05 wt % to about 2 wt %. In an embodiment, the composition comprises lithium acylphosphinate at a concentration from about 0.05 wt % to about 0.1 wt %. In some embodiments, the photoinitiator may be selected based upon the wavelengths of light by which it is activated. For example, in some embodiments, the photoinitiator can have an activation wavelength of from about 200 nm to about 500 nm. In some embodiments, the photoinitiator has an activation wavelength of from about 400 nm to about 700 nm.

The method can further comprise curing the composition to produce a hydrogel material by inducing crosslinking of the polymer material. In some embodiments, this can comprise activating a crosslinking agent in the composition. For example, curing may comprise activating a photoinitiator with light including the activation wavelength for the photoinitiator. In some embodiments, the composition is cured prior to treating a subject (e.g., prior to applying to a subject's tooth).

A hydrogel material prepared as described above may be further processed into discrete pieces or microparticles to facilitate selection and use of a desired amount in therapeutic applications. Such pieces are referred to interchangeably herein as “hydrogel constructs” or “microgels.” In some embodiments, the cured hydrogel may be mechanically reduced to a desired consistency. In some embodiments, the hydrogel material may be formed, e.g., by molding or extrusion, into a solid shape that may then cut into pieces of a selected shape and size or range of sizes. In particular embodiments, the microgels are microscale in size, i.e., measuring less than about 1 mm in two or more dimensions. In some embodiments, microgel size can range from about 1 μm to about 1 mm in two or more dimensions, or more particularly from about 50 μm to about 1 mm, or about 100 μm to about 900 μm, or about 250 μm to about 750 μm.

In some embodiments, the hydrogel material may be formed into microgels having a selected shape. According to an embodiment, this can comprise placing an amount of the uncured hydrogel composition into a shaping device; and curing the hydrogel composition to form a microgel having a shape. In more specific embodiments, the shape comprises one or more of a cylinder, a cuboid, a sphere, a hemisphere, or a prism. In some embodiments, the shaping device used in the method is a mold. According to an alternative specific embodiment the shaping device is a microfluidic channel, for example a channel associated with the printing head of a 3-D printer.

The resulting material may be lyophilized to enhance its stability during storage. Lyophilization can also provide greater ease of handling the bulk material. In some embodiments, lyophilization is carried out by subjecting the material to a gradual freezing rate between 0.5° C./min to 5° C./min. The resulting lyophilized material can also be subsequently subjected to a sterilization process. Such a process may include, for example, the application of gamma-radiation to the lyophilized product, or other approaches suitable for use on such material without affecting its stability.

The lyophilized material may be stored at low temperature, such as at or below about −10° C., until use. Rehydration will allow the material to separate into individual microgels and slowly release dECM components.

In accordance with various embodiments, a method of promoting regeneration of dental tissue can comprise delivering a composition to a cavity in a tooth, where said composition comprises dECM. In some embodiments, the composition can further comprise a polymer material, and can further include a crosslinking agent. In some embodiments, the polymer material comprises a methacrylated gelatin. In some embodiments, the crosslinking agent is a photoinitiator.

In some embodiments, the method may comprise delivering the composition to the tooth in an unset state and then curing the composition to form a hydrogel. Where the composition includes a photoinitiator, the curing step can comprise activating the photoinitiator with light from a light source. In a specific embodiment, the light source is an ultraviolet light source. In another specific embodiment, the light source is a visible light-emitting source.

In some embodiments, the method may comprise delivering the composition as a hydrogel, i.e., in which the polymer material is at least partially crosslinked. The hydrogel may be provided as a bulk microparticulate material comprising discrete hydrogel constructs. In certain embodiments, the hydrogel material is lyophilized and is rehydrated after delivery, which may occur passively by the moisture present in the tooth cavity and/or be accomplished by addition of liquid. In some embodiments, the method may comprise delivering a composition comprising primarily dECM to a tooth cavity, where the composition may not include a polymer material or other carrier. For example, the dECM may be provided as a lyophilized powder.

In accordance with an embodiment, a kit for use in regenerative dentistry can comprise dECM and a crosslinkable polymer material, and can further comprise a crosslinking agent. In some embodiments, the polymer material comprises a methacrylated gelatin. In some embodiments, the crosslinking agent is a photoinitiator.

In a particular embodiment, the kit further comprises instructions for combining the dECM, polymer material, and (if present) crosslinking agent to form a crosslinkable hydrogel composition. In a more specific embodiment, the kit also comprises instructions for delivering and curing the crosslinkable hydrogel composition to form a hydrogel. In another more specific embodiment, the kit further comprises a delivery device for delivering an amount of the crosslinkable hydrogel composition. In some embodiments, the kit comprises a delivery device for delivering an amount of the crosslinked (cured) composition.

In another particular embodiment, at least one of the dECM, polymer material, and crosslinking agent is provided in a container. In a more specific embodiment, the dECM, polymer material, and crosslinking agent are each provided in separate containers.

In another particular embodiment, the dECM is a lyophilized powder.

In accordance with an embodiment, a kit for use in regenerative dentistry comprises a crosslinkable hydrogel composition in a container, where said crosslinkable hydrogel composition comprises dECM, a polymer material, and can further include a crosslinking agent. In some embodiments, the kit comprises a cured composition.

In a more specific embodiment, the kit further comprises instructions for delivering an amount of the crosslinkable hydrogel composition into a tooth and curing the crosslinkable hydrogel composition to form a hydrogel. In some embodiments, the kit comprises instructions for curing the crosslinkable hydrogen prior to delivering into a tooth, and optionally, instructions for shaping, molding or cutting prior to delivering into a tooth.

In another more specific embodiment, the kit further comprises a delivery device for delivering an amount of the crosslinkable hydrogel composition. In a still more specific embodiment, the delivery device is configured to be operably connected to the container for delivering the crosslinkable hydrogel composition directly from the container. In some embodiments, the kit further comprises a delivery device for delivering an amount of the cured composition.

### Example 1. Tooth Procurement and Processing

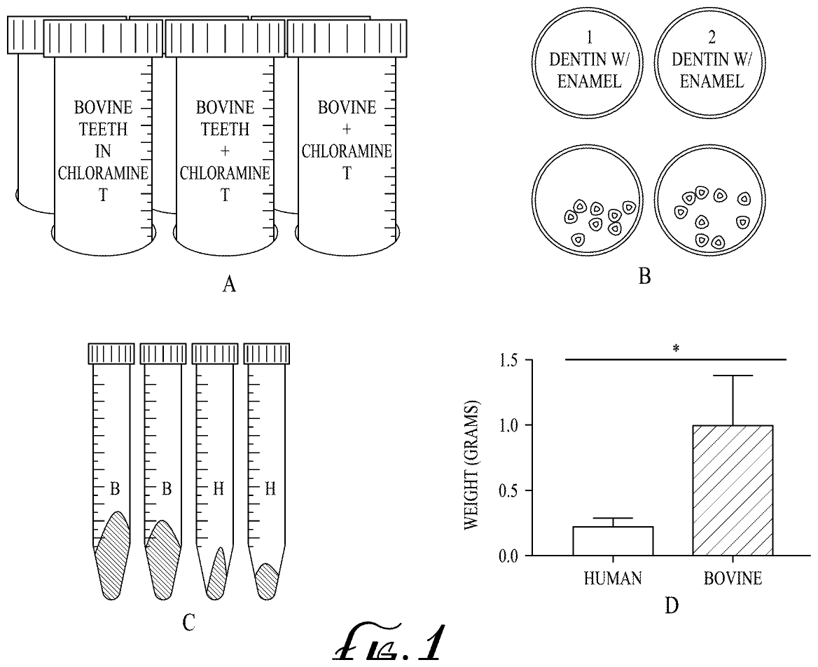

Eighty teeth were collected from Angus and Wagyu 30-month-old slaughtered heifers (FIG. 1 panel A). Some human third molars extracted for orthodontic reasons in clinical settings were used as controls. The teeth were kept in chloramine T solution at 4° C. for 3 hours or no more than one week until processing. Next, soft tissue was mechanically removed using a scalpel blade, and cut into transverse sections using a water-cooled diamond disc saw (FIG. 1 panel B). Pulp tissue was extirpated, and the pulp chamber and root canal were scraped clean with an excavator.

Previous protocols for dentin matrix proteins (DMP) extraction from dentin recommended separation of dentin from enamel with cutters. Herein, to obtain clean dentin slices from each tooth the whole process took around 2 hours, of which half the time was spent separating enamel from dentin. Moreover, it was observed that a considerable amount of dentin was lost at this stage. To scale up the DMP extraction, whether bovine enamel proteins would interfere with the biological properties of the DMP extract and whether processing of the whole tooth would reduce the processing time by ˜50% were tested. To this end, matrix proteins from bovine tooth slices with intact enamel and dentin were extracted to compare against proteins extracted from human dentin alone.

The first step for extracting DMP was to crush the tooth slices into a fine powder using a percussion freezer mill (SPEX 6700 Freezer/Mill®, SPEX® SamplePrep, Metuchen, NJ, USA) (FIG. 1 panel C) cooled with liquid nitrogen to prevent protein denaturation. To obtain biological replicates for this study, each tooth was sliced and ground separately. It took around 2 hours to grind all tooth slices and particular care was taken to constantly keep the liquid nitrogen level at 50% or above to prevent loss or denaturation of proteins. Next, the powdered dentin was separated using a 60 mesh sieve (<0.251 mm²) and the resulting powder was weighed (FIG. 1C). Bovine teeth yielded 3-5 times more powder than human teeth (Student's t-test, p=0.01) (FIG. 1 panel D).

### Example 2. DMP Extraction

To extract the DMP, two extraction solutions each containing a different combination of protease inhibitors were tested: Solution A—10% ethylenediaminetetracetic acid (EDTA) in Tris buffer, pH 7.2 (Sigma) supplemented with 1 μl/ml of Halt protease inhibitor cocktail (Thermo Scientific, Hampton, NH, USA); and Solution B—10% EDTA in distilled water, pH 7.2 with the addition of the protease inhibitors 10 mM n-ethylmalemide (Sigma), and 5 mM phenyl-methyl-sulphonyl fluoride (PMSF) (Sigma).

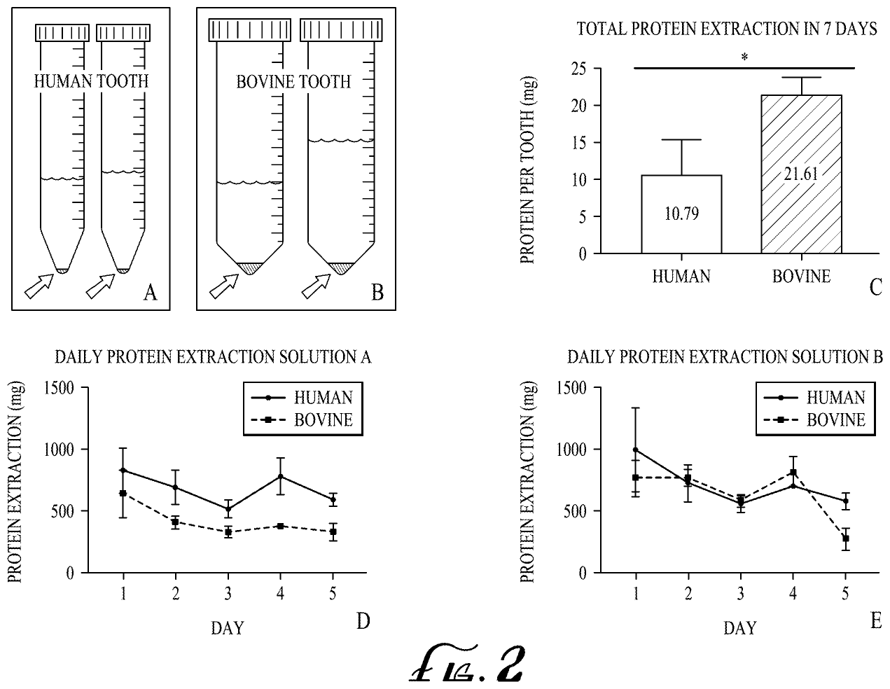

The powder from each tooth was equally split into two centrifuge tubes and either extraction solution A or B, was added to each in a 1:4 (w/v) ratio. Extractions were performed under constant agitation for 7 days at 4° C. Every 24 h, the tubes with the powder and respective solutions were centrifuged at 3000 rpm for 10 minutes at 4° C. and the supernatants were collected and stored separately at −80° C. Human dentin powder precipitates were smaller than bovine pellets (FIG. 2 panels A, B). After 7 days, the supernatants were dialyzed against distilled water using 12-14 kDa dialysis tubing at 4° C. for 5 days with at least two water changes per day. The resultant dialysates were flash frozen in 50 ml centrifuge tubes using liquid nitrogen, lyophilized for 48 h and stored at −20° C. or −80° C. prior to use.

The total protein concentration from the extracts was measured with a Pierce BCA Protein Assay (Thermo Scientific) prepared following the manufacturer's protocol. It was observed that while bovine extracts had an average of 21.61 mg of total protein after 7 days, the human extracts presented only 10.79 mg (Student's t-test, p=0.03) (FIG. 2 panel C). Regarding the efficacy of extraction of the different solutions, there was no statistical difference between Solutions A and B during the timeframe of these experiments (Students t-test: human p=0.7, bovine p=0.08) (FIG. 2 panels D, E).

### Example 3. Proteomic Characterization of Bovine DMP Extracts

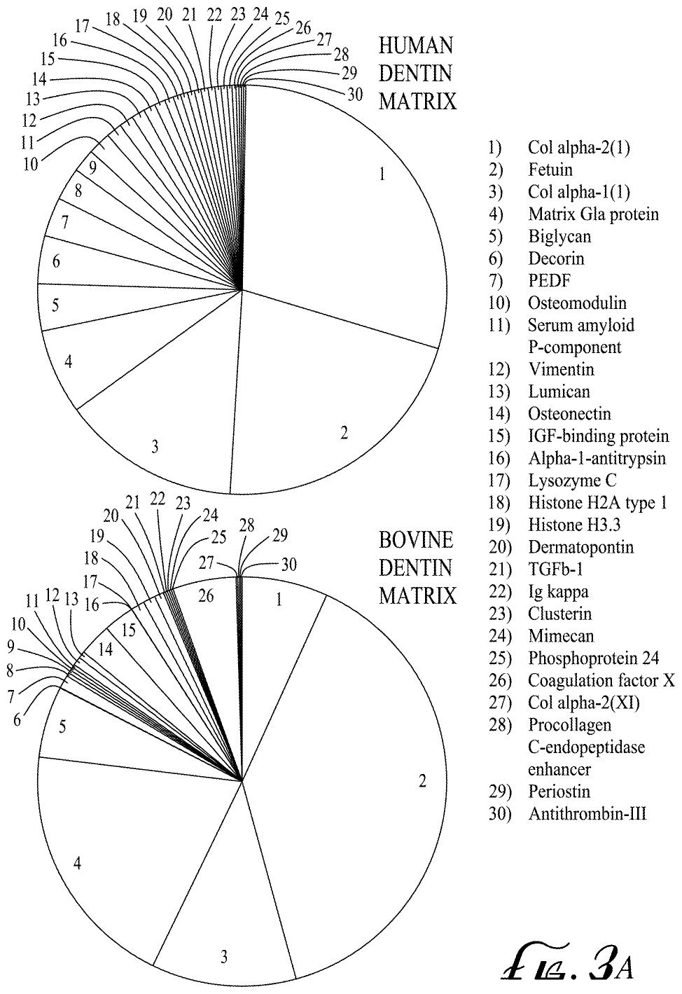

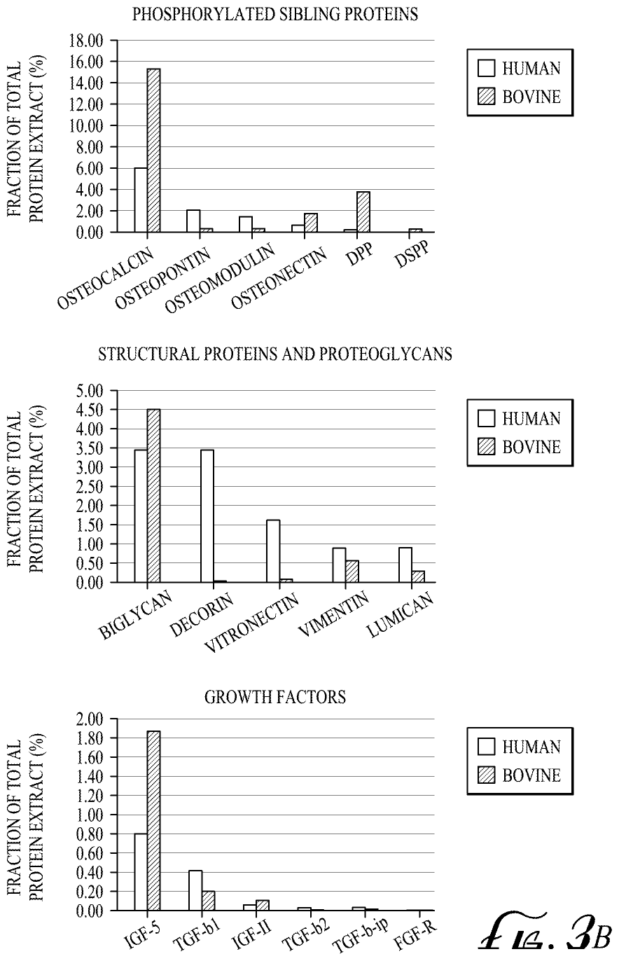

The human (n=4) and bovine extracts (n=4) were analyzed to characterize the total yield and specific concentrations of the pool of molecules. A peptide assay was performed on individual samples to estimate peptide recovery and equal amounts (3 μg) of peptides were pooled from every sample of the same species. Four μg of pooled sample of each species was injected into an Orbitrap Fusion™ mass spectrometer (Thermo Scientific) and run with 240 min liquid chromatograph-mass spectrometry method. Protein identification was performed with CoMeT/PAWS and MaxQuant was used to get intensity-based absolute quantification (iBAQ) feature-based protein intensity. Finally, BLAST was used to determine human and bovine ortholog relationships. After removal of keratin and hemoglobin contaminants, 198 bovine proteins and 126 human proteins were identified. Interestingly, 104 (83%) of the total identified human proteins matched with 50% or greater strength with the bovine proteins. Moreover, these good orthologs accounted for 97% of the total iBAQ signal (FIG. 3A). Proteomic comparisons were made for 30 most abundant human dentin proteins and respective orthologs in bovine dentin matrix extracts. The top 30 proteins detected in human extracts have good orthologs with bovine extracts, but the relative proportion between species is different.

The top matching proteins could be grouped according to their respective biological function into three main groups: (i) phosphorylated SIBLING proteins, (ii) structural proteins and proteoglycans and (iii) growth factors. As shown in FIG. 3B, the relative amount of these proteins varies according to the species. Bovine dentin matrix contains more osteocalcin, biglycan and insulin like growth factor 5 (IGF-5), whereas human dentin matrix presents higher amounts of osteopontin, osteomodulin, decorin, vitronectin and transforming growth factor beta 1 (TGFb-1).

### Example 4. In Vitro Migration Assay

To investigate the chemotactic effects of the gel, migration and invasion assays were conducted. Comparisons were made between (a) gelatin methacryloyl hydrogels supplemented with of DMP (also referred to hereinafter as “RegendoGEL”) (b) unsupplemented gelatin methacryloyl hydrogels (negative control), (c) gelatin methacryloyl hydrogels with recombinant human platelet-derived growth factor BB (rhPDGF-BB; 10 ng/ml) as a positive control, and (d) mineral trioxide aggregate (MTA) the current gold standard.

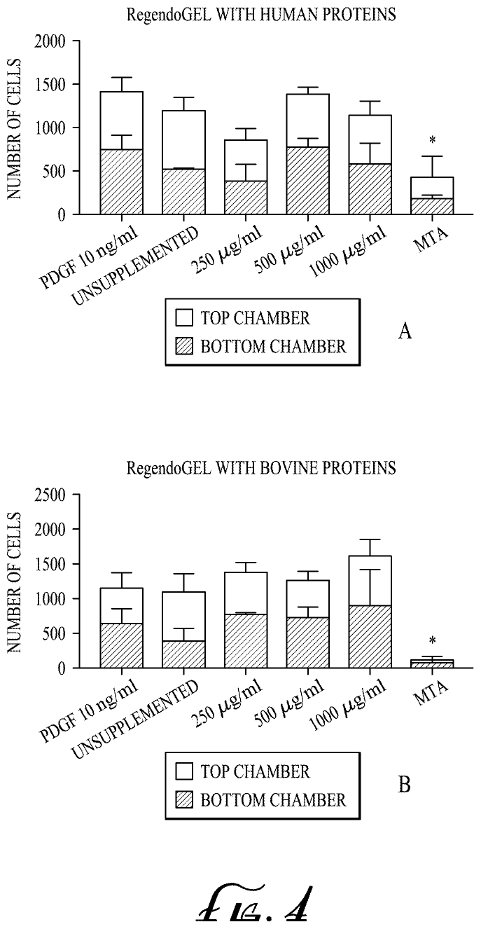

For the migration assay, gelatin methacryloyl hydrogels supplemented with three different concentrations of human DMP (a) 250 μg/ml, (b) 500 μg/ml, and (c) 1,000 μg/ml were tested. The hydrogel constructs were placed in the bottom wells of Boyden chambers and stem cells from apical papilla (SCAPs) were seeded onto the inserts' permeable membrane in a cell density of 3×10⁴ cells/cm². After 24 hours of incubation, the membranes were fixed and stained. Cells that migrated to the bottom side of the membrane and those that remained on the top surface were both quantified. The results (FIGS. 4A, 4B) show that cell migration in the gelatin methacryloyl hydrogel groups with both human and bovine proteins was significantly higher than MTA (ANOVA p=0.001 for human proteins and p=0.02 for bovine proteins). While the chemotaxis obtained with different concentrations of dentin matrix protein was not statistically significant, the greatest amount of cell vitality and migration were obtained with 500 μg/ml supplementation. At least 600% higher migration was obtained with gelatin methacryloyl hydrogels supplemented with 500 μg/ml bovine matrix proteins than with MTA. Comparable levels of cell migration in either human or bovine protein extracts were found.

### Example 5. In Vitro Invasion Assay

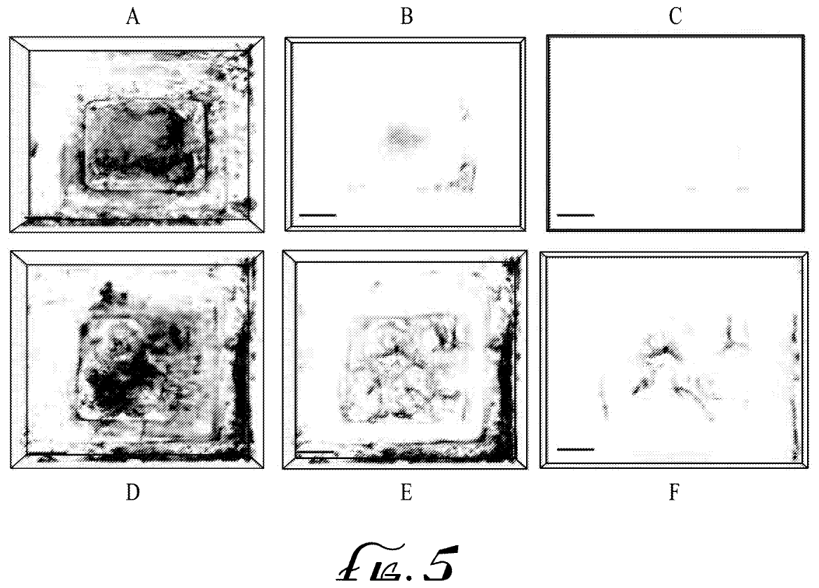

SCAPs (2×10⁴ cells/cm²) were seeded on a 96-well culture plate containing hydrogel constructs. After 3 days of culturing, the cells were fixed with 4% paraformaldehyde and stained with working solution of DAPI and Actin Red and analyzed with a confocal microscope. No invasion was observed after 3 days. Cells seeded on bulk gels formed a monolayer (FIG. 5 panel A) without cell invasion in the middle (FIG. 5 panel B) and bottom (FIG. 5 panel C) of the gel.

Cell migration and invasion in-vivo inside methacrylated gelatin hydrogel has previously been observed. Surprisingly, the cells did not migrate into the gel under these experimental conditions, so other alternatives were tested to address this issue.

An option was to fabricate microgels (for example, squares or circles of ˜500 μm of width and length) to improve cell invasion into the site of repair. Microgels can be fabricated with 3D printed or micromolded hydrogels combining the advantages of good biocompatibility, structural stability of a crosslinked network, tunable mechanical properties and a porous microarchitecture that allows for permeability and more surface for cell attachment as compared to a bulk gel. This option is based on previous results using printed microgels which showed that cells tend to invade more when seeded on top of shaped microgels instead of bulk gels where cells seeded on microgels invaded the core of the construct until the bottom following the microgel's shape (FIG. 5 panels D, E, F). Microgels were then fabricated via 3D printing to investigate whether the depth of invasion would be improved as a function of the hydrogel shape. In short, 7% methacrylated hydrogel with 0.075% LAP photoinitiator containing DMP was added to the printing platform after the print has been loaded into the system. Hydrogel stiffness is controlled via exposure time and allows for material tunability for site specific regeneration.

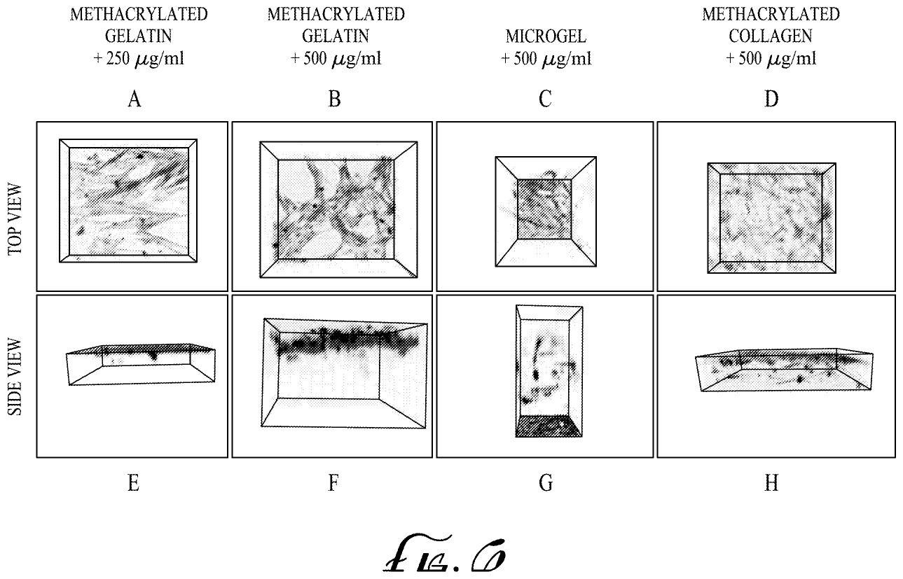

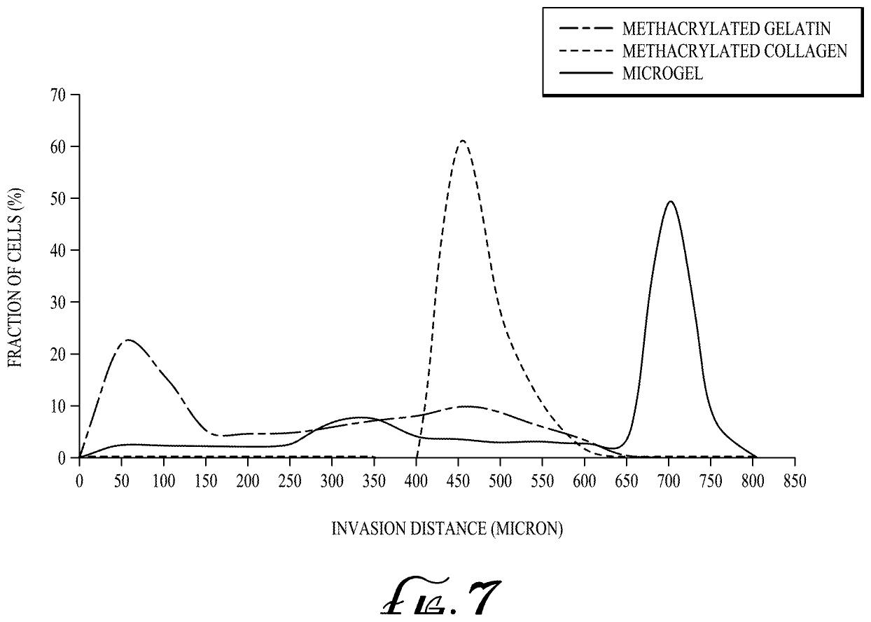

Another option was to exchange the composition for photocrosslinkable collagen. For both conditions the supplementation with 500 μg/ml of bovine DMP was tested. SCAPs (2×10⁴ cells/cm²) were seeded on a 96-well plate containing the hydrogels and after 3 days the cells were fixed with 4% paraformaldehyde, stained for DAPI and Actin Red, and imaged with a confocal microscope (FIG. 6). There was no cell invasion into methacrylated gelatin supplemented with either 250 μg/ml or 500 μg/ml dECM (FIG. 6 panels A, B, E, F). SCAPs attached to the microgels, and the porous nature of the construct enabled cells to migrate and invade relatively long distances (900 μm) (FIGS. 6C, 6G). In a qualitative analysis, cells apparently invaded the methacrylated collagen more than methacrylated gelatin (FIG. 6D, 6H). However, methacrylated collagen degraded faster than methacrylated gelatin, shrinking inside the well plates. Thus, cell invasion was quantified to investigate whether cells were invading the hydrogel or the deformation of the construct was misleading the experiment. At least three biological replicates and cells were quantified using Image J and the results are shown in FIG. 7. To normalize the distance invaded by the cells, the height of each construct was used. Cells seeded on top of methacrylated gelatin (blue line) did not invade the gel. The fraction of ˜20% observed in the graph corresponds to the monolayer formed on the hydrogel surface. On the other hand, 60% of the cells invaded the methacrylated collagen by a range of 450-550 μm. However, as illustrated by the flat orange line up to 350 μm, the hydrogel itself shrunk and most of the cell invasion is restricted to a few μm away from the surface. For the microgels, (gray line) on the other hand, 50% of the cells invaded and distributed nearly homogenously from 50-600 μm, and the remainder accumulated at 650-800 μm of the microgels, possibly due to gravity. With these results further evaluations of microgels were made as the primary mode of fabrication and delivery of DMP for clinical dental pulp regeneration procedures.

### Example 6. Preparation of Microgels for Transportation and Delivery

Microgels were made from the lyophilized DMP described above, and when combined with 7% methacrylated gelatin and 0.075% LAP photoinitiator react to 405 nm wavelength light using a digital light processing 3D printer to make 3D printed microgels of any given microscale size or geometry (FIG. 8 panels A, C). The DMP within the microgels were at a concentration of 500 μg/mL. After the microgels were printed, they were washed with deionized water and subject to freeze drying prior to lyophilization. The lyophilization process can be advantageous, as it enables easier shipping and handling during fabrication and transfer from factory to end-user; while storage is more stable and can potentially be done in a regular fridge or even at room-temperature.

Various experiments were performed to determine the easiest and most effective way of microgel delivery by changing the conditions prior to lyophilization. The outcomes that varied the most includes attachment to the glass slide after they had been printed and rehydrated. FIG. 8 shows different methods performed to distinguish how we could transport the microgels after they had been printed. The lyophilized microgels that were the most successful were the microgels that were removed from the glass slide prior to lyophilization with excess water removed. The lyophilized microgels were stored in a −20° C. freezer until use. Lyophilized microgels are rehydrated in situ where they release DMP through both diffusion and hydrogel degradation. Rehydration allows the microgels to separate into individual microgels and slowly release dECM components that stimulate regeneration of dental pulp cells.

In order to calculate how many microgels were collected, the design that was used was in 5×5 clusters (FIG. 8 panel D). This made it possible to separate the microgels and after lyophilization tweezers were used to pick up the 25 sectioned clumps of microgels and pack them into place (FIG. 8 panels E, F). For long term use, the clinician would be provided enough material per procedure in sterile pre-packaged syringes for direct injection into the target site. An alternative packaging and distributing method could be in a 2, 5, 10, or 25 mg sterilized bottle that the clinician can buy and fill the target site as necessary, as a microparticulate regenerative material. Lastly, the product can be distributed in the form of a paste directly onto the target site via injection from a sterilized pre-packaged tube. The paste is created by mixing RegendoGel mixed with water, as described in additional detail with reference to the Example 15.

### Example 7. In Vitro Migration Assay in Rehydrated Lyophilized Microgels

To investigate the chemotactic effects of the gel, the migration assay was repeated with the lyophilized microgels. Comparisons were made between methacrylated gelatin material supplemented with (a) 500 μg/ml of dentin matrix molecules against (b) unsupplemented methacrylated gelatin material (negative control), (c) rhPDGF-BB (10 ng/ml) (positive control), and (d) MTA (gold standard). This was to ensure 50% more cell migration and invasion than the gold standard, MTA.

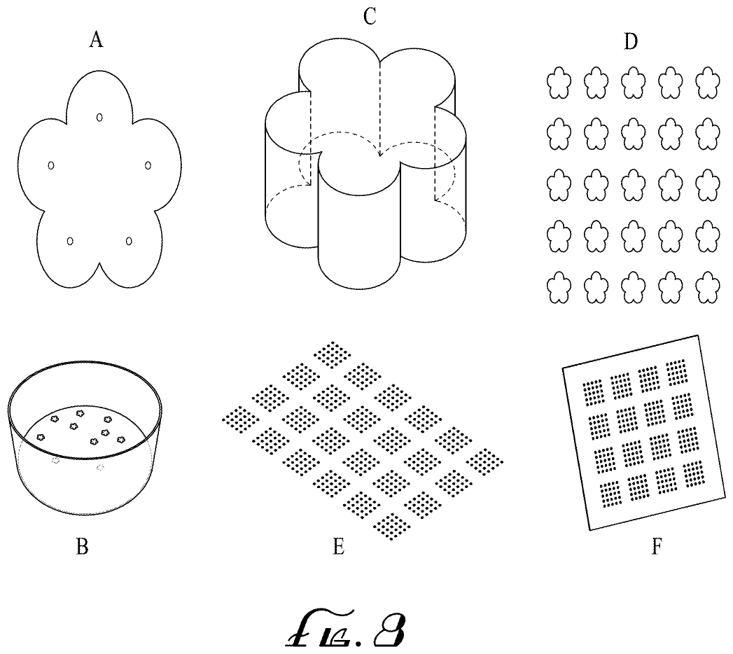

In comparison to the migration assay done with the bulk gel (FIG. 4) which tested the gel supplemented with three different concentrations of DMP (a) 250 μg/ml, (b) 500 μg/ml, and (c) 1,000 μg/ml, for this experiment 500 μg/ml was used as it resulted in the greatest amount of cell vitality and migration previously. The hydrogel constructs were placed in the bottom wells of Boyden chambers and stem cells from apical papilla (SCAPs) were seeded onto the inserts' permeable membrane in a cell density of 3×10⁴ cells/cm². After 24 hours of incubation, the membranes were fixed and stained. Cells that migrated to the bottom side of the membrane and those that remained on the top surface were both quantified. As shown in FIG. 9, gel supplemented with bovine DMP in its lyophilized microgel form also showed significantly greater migration than MTA (ANOVA p=0.001) or unsupplemented methacrylated gelatin.

### Example 8. In Vitro Metabolic Activity Assay

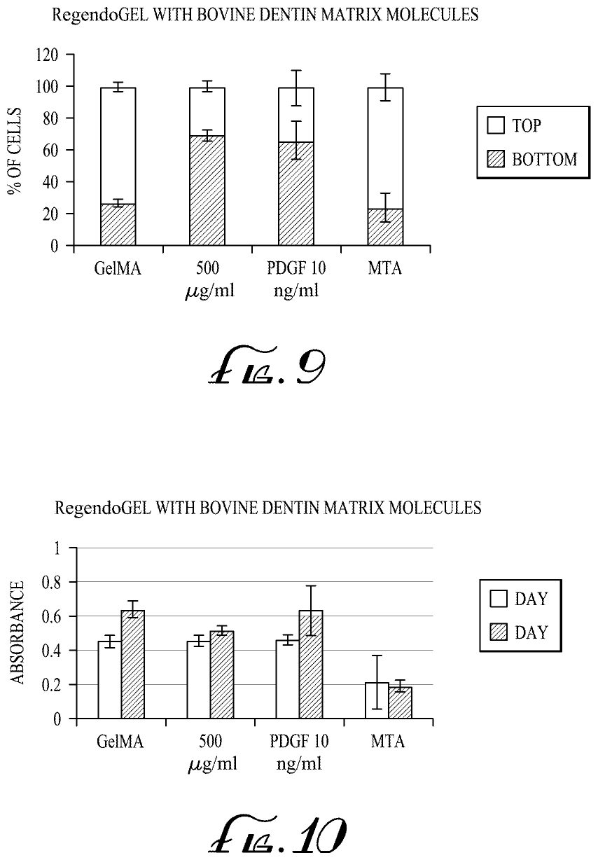

An in vitro 3-[4,5-dimethylthiazol-2-yl]-2,5 diphenyl tetrazolium bromide (MTT) assay was performed to test the metabolic activity of cells cultivated on top of gels supplemented with (a) 500 μg/ml of DMP against (b) unsupplemented (negative control), (c) rhPDGF-BB (10 ng/ml) (positive control), or (d) MTA (gold standard). Absorbance measurements at three and five days (FIG. 10) show that cells cultivated in the presence of methacrylated gelatin show no loss of metabolic activity when compared to the positive control (PDGF) (p>0.05) or unsupplemented methacrylated gelatin, suggesting that there may be no cytotoxic effects on cells. However, with MTA there was 50% loss in metabolic activity which suggests that MTA has more cytotoxic effects on cells.

### Example 9. In Vivo Cytotoxicity Studies in Subcutaneous Rat Models

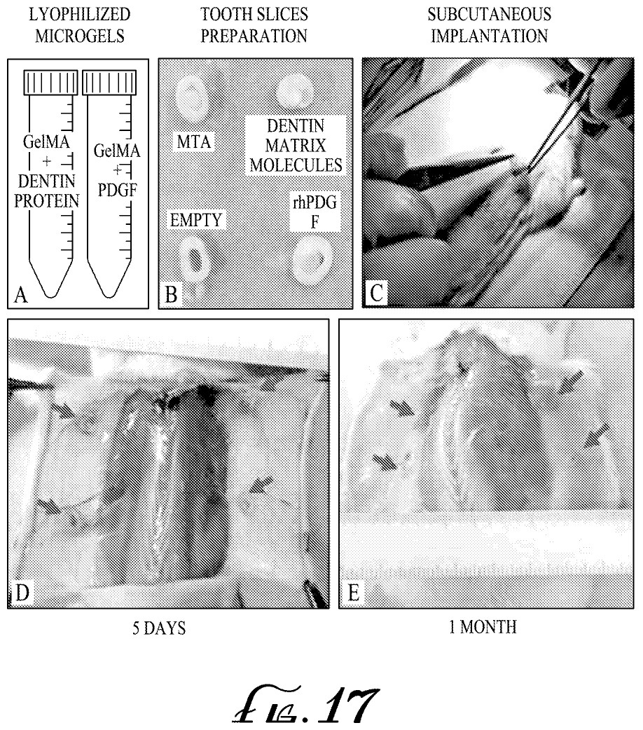

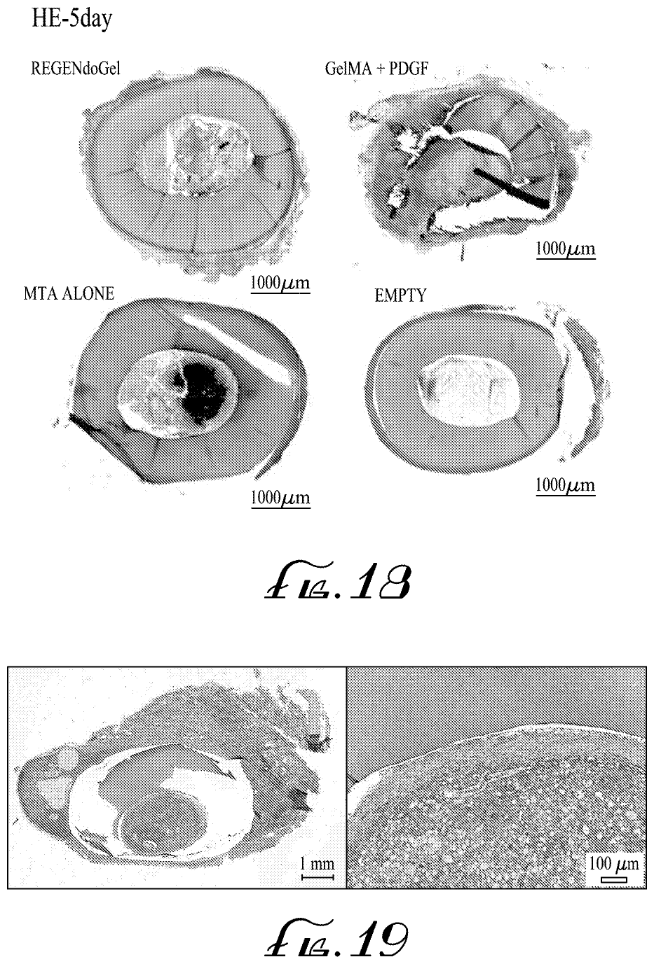

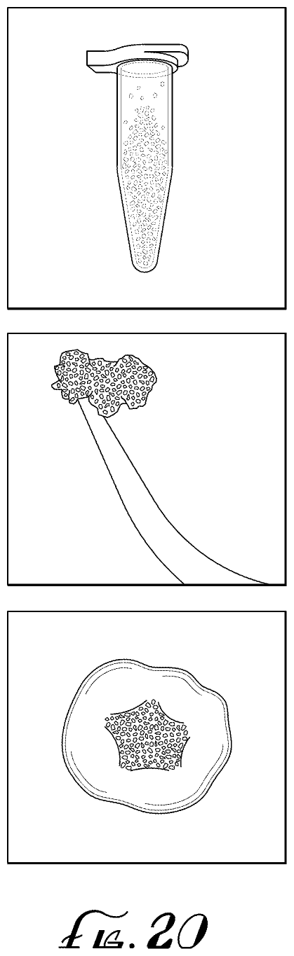

In in vivo cytotoxicity studies the groups that were compared were (a) gel supplemented with 500 μg/ml of DMP with (b) unsupplemented gel (negative control), (c) rhPDGF-BB (positive control) and (d) MTA. The tooth slices were prepared via injection of MTA to enclose one side of the dentin slice and insertion of 25 microgels inside the cavity. The 1 mm tooth slices were then rehydrated with deionized water and implanted in the subcutaneous pockets of the rats (FIG. 17 panels A, B, C). After 5 days (FIG. 17 panel D) and 1 month (FIG. 17 panel E) the samples were retrieved, processed for histology (hematoxylin and eosin (H&E) and von Kossa staining), and immunohistochemistry for von Willebrand factor (vWF) to detect mineral formation and vascularization, respectively, at five days (FIG. 18) and at thirty days (FIG. 19). Formation of odontoblast-like layer near the dentin, highly cellularized pulp-like tissue with extensive blood vessels is observed for those treated with gelatin (FIG. 19, magnified region indicated by inset box).

Immunohistochemical analysis for dentin sialoprotein (DSP) and CD31 can be performed additionally. Cytotoxicity effects can be determined by running tissue samples through a histology microarray panel to screen for inflammatory signaling (Quick Ray, Unitma Co., Ltd.). Cytotoxicity of each formulation may then be graded according to the following scale: 0—No loss in metabolic activity in vitro and no inflammation in vivo; 1—75% loss in metabolic activity in vitro and mild inflammation with a scattering of inflammatory cells, predominately chronic inflammatory cells in vivo, 2—50% loss in metabolic activity in vitro and moderate inflammation with focal accumulations of inflammatory cells but no tissue necrosis in vivo, 3—25% loss in metabolic activity in vitro and severe inflammation in vivo and 4—complete cell death in vitro and abscess formation in vivo.

### Example 10. Long-Term Efficacy

In a long-term efficacy study, 4 mm root fragments are implanted into a bilateral-subcutaneous rat model to study the long-term cell homing and regenerative effects of DMP-supplemented gel compared to MTA. The same analysis described above is performed, except with time points of 30 and 60 days. The samples are retrieved after early and late time points, decalcified and processed for histology and immunohistochemistry. Both vWF and von Kossa staining are performed to detect vascularization and mineral formation respectively. Additionally, samples are stained for DSP and CD31 to detect odontogenic and vasculogenic tissue formation, respectively. Lysed tissue extracts are analyzed using RT-PCR to quantify expression of vasculogenic and odontogenic gene expression such as VEGFR1, VEGFR2, PECAM1, PDGFRB, DSPP1 and DMP-1.

### Example 11. Microgel Construct Using Micro-Sectioning

Microgels were made from the lyophilized dentin matrix molecules described above. The combination of 7% methacrylated gelatin and 0.075% LAP photoinitiator was placed in a mold, exposed to 405 nm wavelength light and sectioned to make microgels (10 μm and 1000 μm in size.) Hydrogel micro sectioning made use of a tissue chopping machine to section preformed sheets of hydrogel into strips in the X direction and cut again in the Y direction to create the final microgels. The hydrogel chopping produced microgels a rate of 9600 microgels/h. This process is readily scalable with use of more than one sectioning instrument. The lyophilized microgels were stored in a −20° C. freezer until use. Rehydration allowed the microgels to separate into individual microgels and slowly release dECM components.

### Example 12. In Vivo Direct Pulp Capping

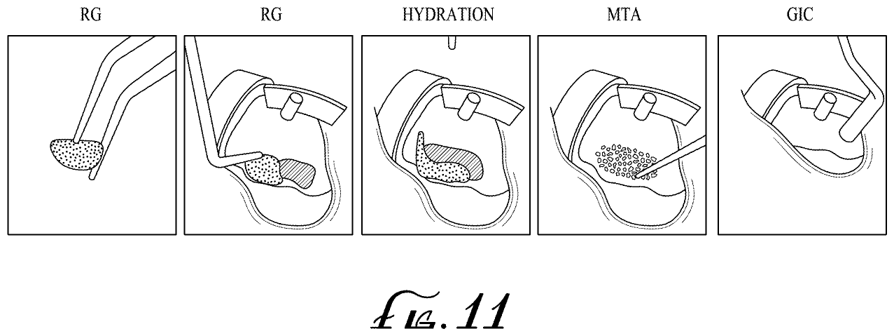

A protocol for direct pulp capping previously described by others was used in this study. In brief, with the aid of a dental microscope (4.5× magnification), cavities were prepared on the occlusal surfaces of the maxillary first molar using a 0.5 mm diameter round bur (KG Sorensen, Sao Paulo, Brazil) in a high-speed handpiece under abundant irrigation. Dental pulp was exposed using an endodontic file (#15 sterile stainless steel; Dentsply Maillefer, Ballaigues, Switzerland) through the remaining thin dentin of each cavity. Bleeding was controlled by pressing sterile paper points for a few seconds. FIG. 11 shows images of the steps of gelatin delivery and pulp capping in a dog. RegendoGEL was delivered into the cavity using a pair of tweezers and placed above the pulp exposure. Hydration of the lyophilized microgels occurred spontaneously after they were placed within the cavity and allowed the separation of the material into individual microgels. After microgel placement, a thin layer of White MTA was placed in all cavities above RegendoGEL, after setting the environment was favorable to adhesion with restorative material. Then, the cavities were sealed using self-etching adhesive (Single Bond Universal—3M ESPER) and restored with composite resin Z350XT (3M ESPER) to allow marginal sealing and decrease the occurrence of marginal infiltration. The cusp tips of the opposing teeth were cut to minimize occlusal forces.

### Example 13. In Vivo Cytotoxicity Studies in a Pulpotomy Rat Model

The direct pulp capping protocol described in Example 12 was performed on young adult male Wistar rats (Rattus norvegicus) weighing 180-220 g. The rats were randomly distributed into six groups (n=15/group) including: (1) negative control (NC) treated with commercial temporary filling material with inert properties (Zinc Oxide, Zinc Sulphate, Calcium Sulphate, Polyvinyl Acetate, Menthol, Dibutylphthalate—(Cotosol, Villevie, Joinville, SC, Brazil); (2) a clinical gold standard treatment with White MTA (Angelus, Londrina, PR, Brazil); (3) unsupplemented microgels comprising GelMA alone (Microgel); (4) microgels supplemented with dentin matrix molecules—500 μg/ml (Microgel+DMM 500 μg/ml); (5) microgels supplemented with dentin matrix molecules—1000 μg/ml (Microgel+DMM 1000 μg/ml); and (6) positive control—microgels supplemented with platelet-derived growth factor (PDGF)—10 ng/ml (Microgel+PDGF 10 ng/ml). Microgels were made according to the method described above in Example 6.

Animals were anesthetized intraperitoneally and sacrificed 4 weeks after surgery. The right maxillary first molar treated teeth and left side maxillary first molar control untreated teeth were harvested, fixed with 10% neutral formalin for 24 hours at 4° C., and demineralized in 10% EDTA/phosphate-buffered saline solution for one month. After trimming, tooth samples were embedded in paraffin, and serial sectioned in the mesiodistal direction at 4 mm-thickness and stained with hematoxylin & eosin. A trained observer with no previous knowledge of the groups performed a blinded histological evaluation for inflammatory cell response, quality of dentin bridge formation, tertiary dentin deposition, pulp tissue organization and hard tissue formation. The percent of tertiary dentin in the dental pulp tissue under the pulp capping treatment region was quantified using Image J. The results of inflammatory cell infiltration and hard tissue formation were analyzed using the Statistical Package for the Social Sciences (SPSS; version 20.0) and the frequencies of each parameter were compared by Pearson's chi-square test (p<0.05). For the release assay two-way ANOVA was used with Tukey as post-hoc (α=0.05).

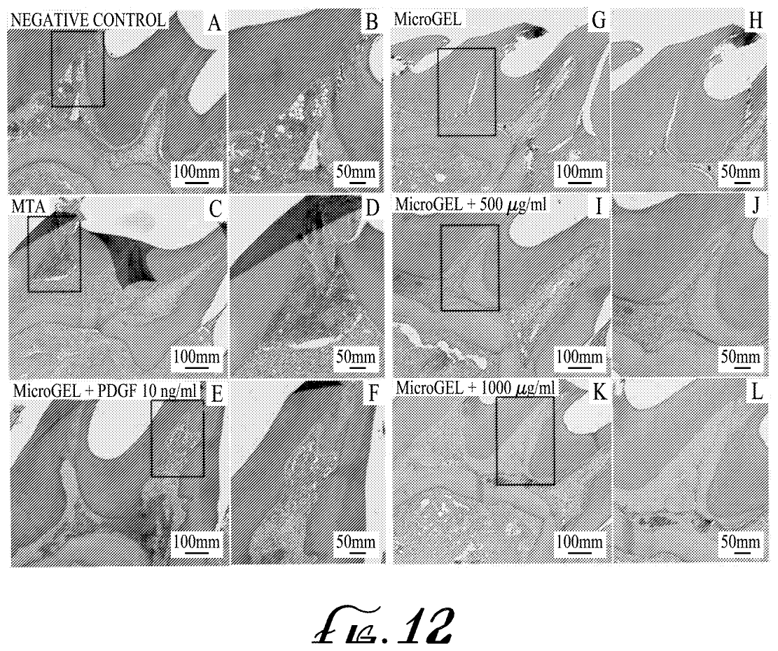

After 4 weeks of treatment, morphological analysis of the dental pulp showed that most negative control group specimens exhibited dental pulp necrosis and loss of overall dental pulp tissue organization (FIG. 12 panels A, B). Moreover, one third of these samples showed what appeared to be adipocytes close to the injury site (FIG. 12 panel B). The MTA group exhibited severe inflammatory infiltrate below the material (FIG. 12 panels C, D). All microgel samples (unsupplemented and supplemented) showed reestablishment of an organized odontoblast layer adjacent to the microgel, comparably lower levels of inflammation as compared to MTA, and the presence of newly deposited reparative dentin (FIG. 12 panels E-J). Microgel+PDGF 10 ng/ml groups showed large blood vessels filled with erythrocytes in the regenerated pulp (FIG. 12 panels E, F). The level of dental pulp tissue organization among the groups was quite similar, except for the negative control group which appeared very disorganized (FIG. 12). More extensive amounts of tertiary dentin were observed in all microgel treated groups as compared to other groups, and in some microgel treated specimens a healthy pulp tissue was observed adjacent to treated defect site (FIG. 12).

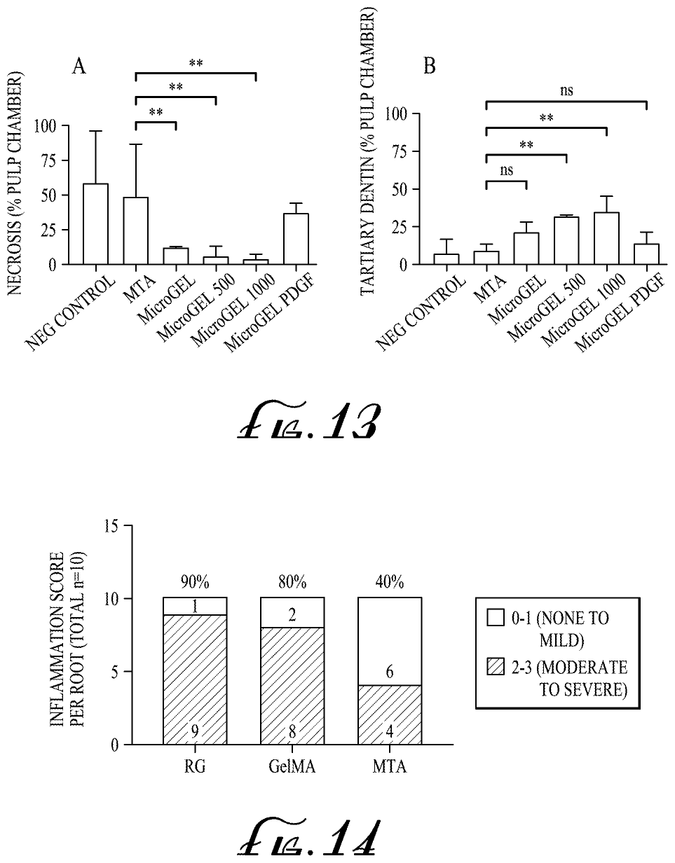

Image J was used to quantify the percent necrosis in dental pulp tissue located under the pulp capping treatment (FIG. 13 panel A) as compared to the total area of the dental pulp. Microgel, Microgel+DMP 500 μg/ml, Microgel+DMP 1000 μg/ml groups exhibited the lowest percent necrosis. Conversely, necrosis was increased in samples from the negative control group, and MTA and PDGF+DMP 1000 μg/ml treated groups. Microgel+DMP 500 μg/ml and Microgel+DMP 1000 μg/ml groups had significantly more newly formed tertiary dentin as compared to MTA after one month (FIG. 13 panel B).

### Example 14. In Vivo Cytotoxicity Studies in a Pulpotomy Dog Model

To determine the efficacy of RegendoGEL in tertiary dentin formation and lack of cytotoxicity in vivo, a pulpotomy dog model study was performed consisting of the following experimental groups: RegendoGEL; GelMA alone as a negative control; and MTA as a positive control. Microgels were made according to the method described above in Example 11. Experimental animals were euthanized on days 5 and 70 to evaluate inflammation and tertiary dentin formation, respectively. Samples were harvested from both timepoints and microCT and histological analyses for routine H&E staining were performed. Inflammatory scores were based on the ISO recommendation, which is 0 for no inflammation, 1 for mild, 2 for moderate and 3 is for the presence of severe inflammatory infiltrate.

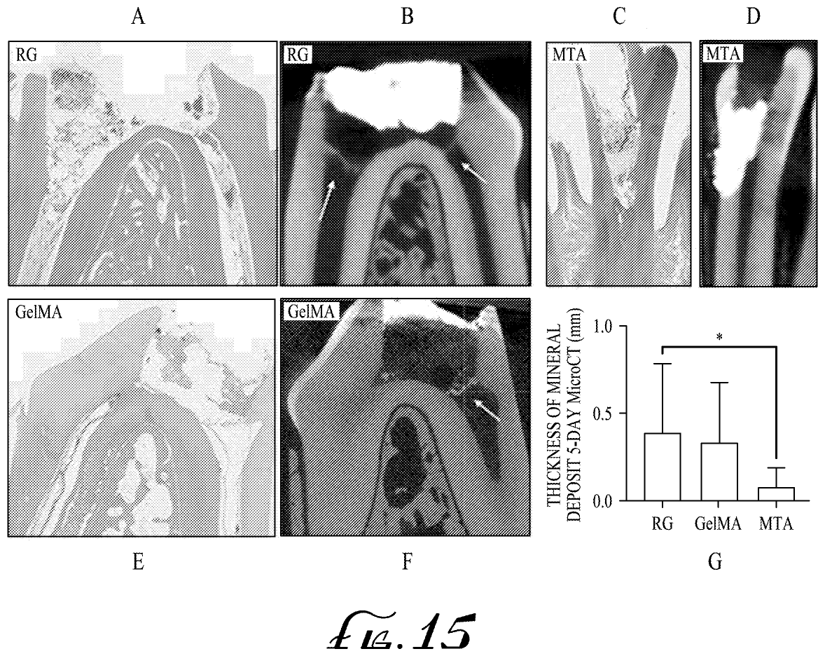

Only the roots with enough dental pulp tissue were considered in this analysis (n=10). Pulpotomy tooth samples that received RegendoGEL or GelMA showed mostly mild to no inflammation (90% and 80% respectively) while MTA was highly inflammatory to the pulp, with 60% of the samples showing moderate to severe inflammatory infiltrate in at least some areas of the dental pulp (FIG. 14).

### Example 15. Dentin Characterization Study

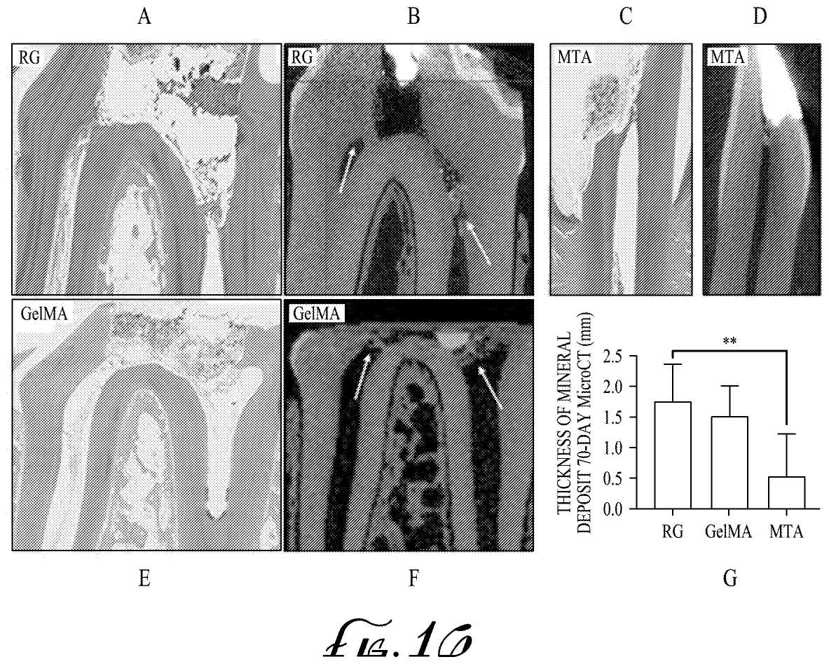

MicroCT and histological data were compared between treatment groups at 5-different time points. Comparison of the microCT and histological data in 5-day samples showed correlation of the tertiary dentin in histological sections with the mineralized deposits present in the microCT (FIG. 15). Tertiary dentin deposition was detected in 66% of RegendoGEL-treated teeth, and in 68% of GelMA-treated teeth. Such deposition was characterized by either linear mineral deposits, tubular structures or amorphous mineralized masses. Conversely, only 28% of MTA-treated teeth presented mineral deposits or thin mineralization underneath the material (FIG. 15). A quantitative analysis of the mineral deposits demonstrated that the mineral thickness was similar between RegendoGEL (0.61 mm±0.31) and GelMA (0.52 mm±0.27, mean and SD), but higher than in MTA-treated teeth (0.2 mm±0.08, mean and SD, one-way ANOVA, Tukey post-hoc, alpha 0.05) (FIG. 15).

MicroCT analysis of 70-day samples showed an increased difference between RegendoGEL, GelMA and MTA regarding mineral deposition. For multiradicular teeth, each root (mesial and distal) canal was analyzed independently. Interestingly, 100% of the teeth treated with RegendoGEL had dentin bridges or mineral deposition formed in at least one root canal, contrasting greatly with MTA-treated teeth that had 50% of mineral deposition or dentin bridge formation (FIG. 16). Quantification of the thickness of the mineral deposition via microCT after 70 days demonstrated that RegendoGEL-treated pulps formed thicker mineral deposition, characterized mostly by a dense dentin bridge at the opening of the root canal (1.7 mm±0.6, mean and SD), followed by GelMA-treated pulps with 1.5 mm±0.4 (mean and SD) (FIG. 16). MTA-treated teeth had fewer mineral deposits, and although some teeth showed a complete dentin bridge formation, such bridge had an average thickness of 0.5 mm±0.2 (mean and SD), 3 times thinner than the dentin bridge induced by RegendoGEL (FIG. 16).

### Example 16. RegendoGel (RG) Microparticles in a Paste Form Factor for In Vivo Study

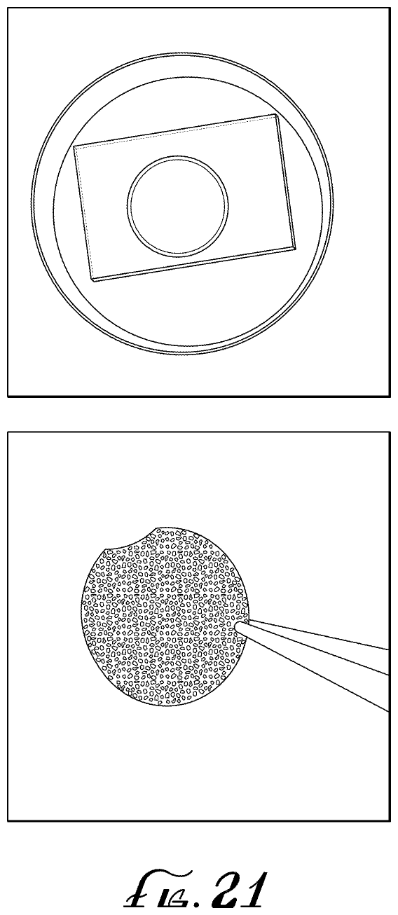

With reference to Example 6, the following passages describe the process for application of microparticles in a paste form. First, an Eppendorf tube or other container of the RG microparticles is flicked or shaken to remove any clumps and position microparticles in the bottom of the tube (FIG. 20, top). As described previously, the size of the particles is in a range from about 10 to about 1000 microns, in some embodiments. Second, the Eppendorf vial containing RG-MPs is opened carefully so as to not lose any MPs. Third, one drop of saline solution or DI water is added to the MPs. Fourth, the drop is gently mixed with an applicator. The mix turns into a jelly-like material that is easy to pick up (FIG. 20, middle). Fifth, wet microparticles are carefully placed into the pulp chamber, with care taken to not condense the placed material (FIG. 20, bottom). Sixth, the tooth is covered with a restorative material (MTA with Ketac, or directly with Ketac) as per protocol.

Skilled persons will appreciate that the paste could dry out, in which case the paste may be deployed in the dry form, and that it may be more stable over time.

### Example 17. RegendoGEL Membranes

With reference to the previously described methods and formulations for creating the microparticle form factor of RegendoGEL, a method to produce a second form factor, i.e., a membrane, is described as follows. In this example, the membrane is the RegendoGEL material prior to sectioning. Initially, it is noted that the thickness of the membranes may be in the microns range (for example, between 1 and 1,000 microns) in some instances approximately 450 microns, or approximately 400 microns, or approximately 350 microns, or approximately 350 microns to 450 microns. The membranes can be made for chopping them up to make the microparticles (or larger particles) form of RegendoGel.

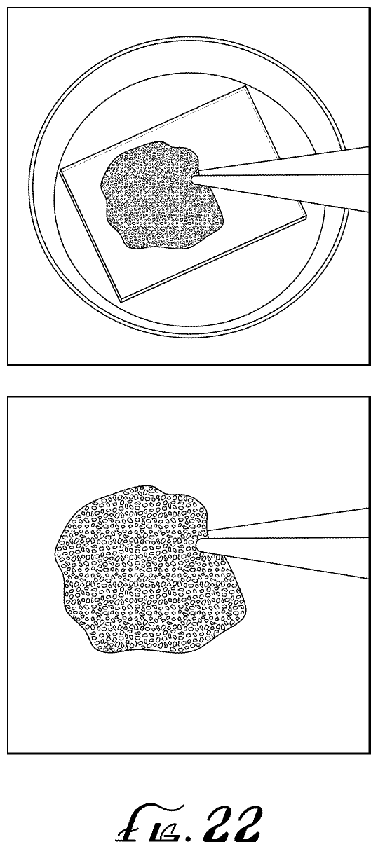

Two acrylic spacers (60×20×0.45 mm) with the thickness of 450 μm, are positioned along the sides of the PDMS window of an Ember printer tray. A 200 μl aliquot of pre-warmed GelMA/DPBS/LAP solution is dispensed into the PDMS window of the Ember printer tray, between the two spacers. A glass slide is placed atop the spacers and the aliquoted GelMA/DPBS/LAP solution. A 405 nm wavelength light is used to light cure the solution for 25 seconds, which gels into a sheet. The gel sheet is washed with sterile DPBS and transferred onto a piece of PDMS using a razor blade (FIG. 21, top). The PDMS and gel sheet is then placed into a petri dish and freeze dried for 24 hours. After freeze drying, the gel is lyophilized for 24 hours and evaluated for handling properties (FIG. 21, bottom).

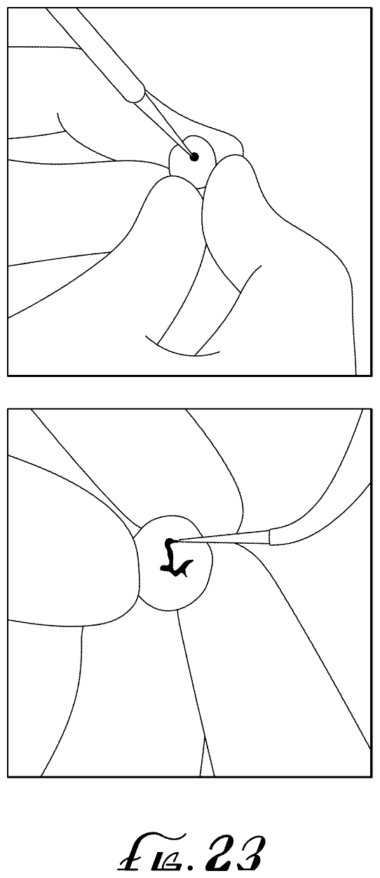

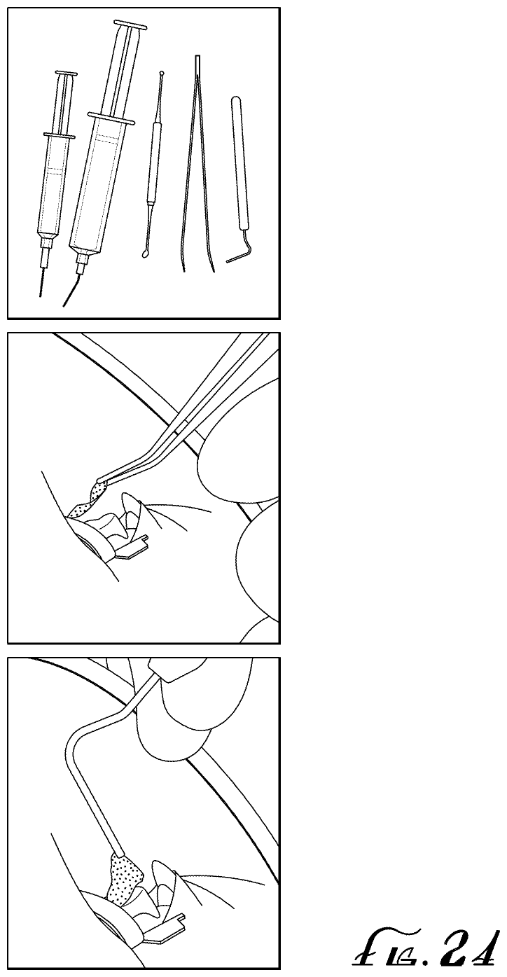

The aforementioned technique is also suitable for making a thinner membrane, which the inventors believe is more biologically efficient to release DMMs into the pulp chamber. For example, membranes of 350 μm thickness were created without compromising the handling properties of the membrane (FIG. 22). The 350 μm thick RegendoGEL membrane may then be cut to an appropriate size and implanted into the pulp chamber of an extracted human molar (see, e.g., FIG. 23).

### Example 18. RegendoGel Membrane for In Vivo Dog Study

The following passages described steps for RG membrane application. First, remove RG membrane from sterilization package and place on cutting board. Second, cut a piece of RG membrane to fit the size of defect pulp chamber floor (FIG. 24, middle). Third, using tweezers (or tool of clinician's choice), carefully place the RG membrane into the prepared cavity on top of the healthy dental pulp (FIG. 24, middle). Fourth, use an instrument (plugger) to gently push the membrane down into the pulp chamber in contact with the dental pulp (FIG. 24, bottom). Fifth, cover with restorative material (MTA with Ketac, or directly Ketac).

### Example 19.

Embodiments disclosed herein may also be provided in combination with the teachings of PCT/US2017/064312, published as WO/2018/102750, and U.S. Pat. No. 11,278,474, both of which are incorporated herein by reference in their entireties.

It will be apparent to those having skill in the art that many changes may be made to the details of the above-described embodiments without departing from the underlying principles of the invention. The scope of the present invention should, therefore, be determined only by the following claims.

1. A composition for promoting regeneration of dental tissue, comprising a dentin extracellular matrix material (dECM).

2. The composition of claim 1, wherein the dECM comprises dentin matrix proteins having an origin selected from human, ovine, porcine, and bovine.

3. The composition of claim 2, wherein the dECM comprises human dentin matrix proteins.

4. The composition of claim 2, wherein the dECM comprises bovine dentin matrix proteins.

5. The composition of claim 2, wherein the dECM comprises porcine dentin matrix proteins.

6. The composition of any one of claims 1 to 5, wherein the dECM is present in a concentration of from about 100 μg/ml to about 1,200 μg/ml.

7. The composition of claim 6, wherein the concentration is from about 250 μg/ml to about 600 μg/ml.

8. The composition of any one of claims 1 to 7, wherein the dECM is dispersed in a polymer material, and wherein said polymer material is crosslinkable, biocompatible and biodegradable.

9. The composition of claim 8, further comprising a crosslinking agent.

10. The composition of claim 9, wherein the crosslinking agent is a photoinitiator.

11. The composition of claim 10, wherein the photoinitiator is selected from lithium phenyl-2,4,6-trimethylbenzoyl phosphinate, lithium acylphosphinate, 2-Hydroxy-4′-(2-hydroxyethoxy)-2-methylpropiophenone and 2-hydroxy-1-(4-(hydroxyethoxy)phenyl)-2-methyl-1-propanone.

12. The composition of claim 10, wherein the photoinitiator has an activation wavelength of from about 200 nm to about 500 nm.

13. The composition of claim 10, wherein the photoinitiator has an activation wavelength of from about 400 nm to about 700 nm.

14. The composition of any one of claims 10 to 13, wherein the photoinitiator is present at a concentration of from 0.05 wt % to 2 wt %.

15. The composition of any one of claims 8 to 14, wherein the polymer material comprises a functionalized gelatin.

16. The composition of claim 15, wherein the polymer material comprises a methacrylated gelatin.

17. A hydrogel construct comprising the composition of any one of claims 8 to 16, wherein the dECM or the polymer are at least partially crosslinked to one or more other components of the composition.

18. The hydrogel construct of claim 17, wherein the hydrogel construct is lyophilized.

19. The hydrogel construct of claim 17 or claim 18, exhibiting at least one shape selected from a cylinder, a cuboid, a sphere, a hemisphere, and a prism.

20. The hydrogel construct of any one of claims 17 to 19, having a volume from about 0.05 mm³ to about 1 mm³.

21. The hydrogel construct of any one of claims 17 to 19, having size of about 1 μm to about 1 mm in two or more dimensions.

22. A bulk material comprising a plurality of the hydrogel construct of any one of claims 17 to 21.

23. A method of promoting regeneration of dental tissue, comprising delivering to a surface of a tooth the composition any one of claims 1 to 7 as a paste or a portion of a membrane.

24. A method of promoting regeneration of dental tissue, comprising the steps:
delivering the composition of any one of claims 7 to 16 to a surface of a tooth; and
curing the hydrogel composition.

25. A method of promoting regeneration of dental tissue, comprising delivering at least one hydrogel construct of any one of claims 17 to 21 to a surface of a tooth, optionally wherein the hydrogel construct is a least partially cross-linked or cured prior to delivering the hydrogel to the surface of the tooth.

26. A method of making a material for promoting regeneration of dental tissue, comprising:
providing a dECM; and
dispersing the dECM within a polymer material to make a hydrogel composition, wherein said polymer material is crosslinkable, biocompatible and biodegradable.

27. The method of claim 26, further comprising combining a crosslinking agent with the dECM and the polymer material.

28. The method of claim 27, wherein the crosslinking agent is a photoinitiator.

29. The method of claim 28, wherein the photoinitiator is selected from lithium phenyl-2,4,6-trimethylbenzoyl phosphinate, lithium acylphosphinate, 2-Hydroxy-4′-(2-hydroxyethoxy)-2-methylpropiophenone and 2-hydroxy-1-(4-(hydroxyethoxy)phenyl)-2-methyl-1-propanone.

30. The method of claim 28, wherein the photoinitiator has an activation wavelength of from about 200 nm to about 500 nm.

31. The method of claim 28, wherein the photoinitiator has an activation wavelength of from about 400 nm to about 700 nm.

32. The method of any one of claims 26 to 31, wherein the dECM comprises dentin matrix proteins having an origin selected from human, ovine, porcine, and bovine.

33. The method of claim 32, wherein the dECM comprises human dentin matrix proteins.

34. The method of claim 32, wherein the dECM comprises bovine dentin matrix proteins.

35. The method of claim 32, wherein the dECM comprises porcine dentin matrix proteins.

36. The method of any one of claims 26 to 35, wherein the polymer material comprises a functionalized gelatin.

37. The method of claim 36, wherein the polymer material comprises a methacrylated gelatin.

38. The method of any one of claims 26 to 37, further comprising curing the hydrogel composition to form at least one hydrogel construct.

39. The method of claim 38, comprising imparting a shape to the hydrogel construct, wherein the shape is selected from a cylinder, a cuboid, a sphere, a hemisphere, and a prism.

40. The method of claim 39, comprising introducing the hydrogel composition in a shaping device configured to impart the shape to the hydrogel construct.

41. The method of claim 40, wherein the shaping device is a mold.

42. The method of claim 40, wherein the shaping device is a microfluidic channel.

43. The method of claim 38, comprising mechanically disaggregating the hydrogel composition after curing to form at least one hydrogel construct.

44. The method of any one of claims 38 to 43, further comprising lyophilizing the hydrogel construct.

45. A kit for use in regenerative dentistry, comprising ingredients including a dECM and a polymer material, wherein said polymer material is crosslinkable, biocompatible and biodegradable.

46. The kit of claim 45, wherein the ingredients further include a crosslinking agent.

47. The kit of claim 45 or 46, further comprising instructions for combining the ingredients to form a hydrogel composition.

48. The kit of any one of claims 45 to 47, further including instructions for delivering and curing the hydrogel composition.

49. The kit of any one of claims 45 to 48, further comprising a delivery device for delivering an amount of the hydrogel composition.

50. The kit of any one of claims 45 to 48, wherein at least one of the ingredients is provided in a container.

51. The kit of claim 50, wherein the ingredients are each provided in separate containers.

52. The kit of claim 46, wherein the crosslinking agent is a photoinitiator.

53. The kit of any one of claims 45 to 52, wherein the dECM is a lyophilized powder.

54. A kit for use in regenerative dentistry, comprising a hydrogel composition in a container, said hydrogel composition comprising dECM and a polymer material, wherein said polymer material is crosslinkable, biocompatible and biodegradable.

55. The kit of claim 54, wherein the hydrogel composition further comprises a crosslinking agent.

56. The kit of claim 54 or 55, further comprising instructions for delivering an amount of the hydrogel composition into a tooth and curing the hydrogel composition to form a hydrogel.

57. The kit of any one of claims 54 to 56, further comprising a delivery device for delivering an amount of the hydrogel composition.

58. The kit of claim 57, wherein the delivery device is configured to be operably connected to the container for delivering the hydrogel composition directly from the container.

59. The kit of any one of claims 54 to 58, wherein the crosslinking agent is a photoinitiator.

60. The kit of claim 59, wherein the photoinitiator is selected from lithium phenyl-2,4,6-trimethylbenzoyl phosphinate, lithium acylphosphinate, 2-Hydroxy-4′-(2-hydroxyethoxy)-2-methylpropiophenone and 2-hydroxy-1-(4-(hydroxyethoxy)phenyl)-2-methyl-1-propanone.

61. The composition of any one of claims 17-21, wherein the dECM or the polymer are crosslinked to one or more other components of the composition.

62. A method of promoting regeneration of dental tissue, comprising delivering the composition of claim 61 to the surface of a tooth.

63. The method claim 25 or 61, wherein the hydrogel construct or composition is delivered in the form of a sheet, or in solution as droplets, or in the form of a microgel.

64. The method of claim 62, comprising imparting a shape to the hydrogel construct or composition prior to delivering the hydrogel construct or composition to the surface of the tooth, wherein the shape optionally is imparted by cutting.

65. The kit of any of claims 45-60 further comprising instructions for curing the hydrogel and for delivering an amount of the cured hydrogel to a tooth.

66. A kit for use in regenerative dentistry, the kit comprising: (i) a container comprising a composition comprising dECM and a polymer material that are biocompatible and biodegradable; and optionally (ii) a containing comprising a crosslinking agent.

67. The kit of claim 66, comprising a container comprising a crosslinking agent.

68. The kit of claim 66 or 67, wherein the crosslinking agent is in the same container as the dECM and the polymer material.

69. A kit for use in regenerative dentistry, the kit comprising: a container comprising a composition comprising dECM and a polymer material that are biocompatible and biodegradable, wherein one or both of the dECM and polymer material are crosslinked, and optionally the composition is in the form of a cured hydrogel.

70. The kit of any one of claims 66-69 wherein the container comprising the dECM and the polymer material comprises a mold.

71. The kit of claim 69 or 70, wherein the cured hydrogel is in the form of a sheet or droplets.

72. The kit of any of claims 69-71, wherein the cured hydrogel is in the form of a microgel.
